(19) United States
(12) Patent Application Publication
(10) Pub. No.: US 2007/0207054 A1
(43) Pub. Date: Sep. 6, 2007

(54) Sterilizing apparatus and method

(76) Inventor: Terrence R. Langford, Tucson, AZ (US)

Correspondence Address: QUARLES & BRADY LLP, ONE SOUTH CHURCH AVENUE, SUITE 1700, TUCSON, AZ, 85701-1621, US

(21) Appl. No.: 11/367,787

(22) Filed: Mar. 3, 2006

(51) Int. Cl. A61L 2/00

(52) U.S. Cl. 422/28; 422/33; 422/26; 422/34; 422/292

(57) ABSTRACT

Methods and apparatus for liquid, gas, and gas plasma sterilization of items. An item is located within a sterilizing apparatus and sterilized through the introduction of fresh sterilant at least once after an initial amount of sterilant is introduced into the apparatus. Alternatively, an item is contained within sterile packaging, positioned within a baffle, and sterilized through the flowing, including bi-directional flowing, of fresh sterilant. The apparatus includes one or multiple chambers within with cleaning and sterilizing take place.

### BACKGROUND OF THE INVENTION

1. Field of the Invention

This invention relates generally to improved apparatus and methods for sterilizing both packaged and unpackaged items, and, more particularly, to apparatus and methods that involve dynamically flowing fresh sterilant through a chamber containing the item to be sterilized.

2. Description of the Related Art

The reprocessing (i.e., cleaning and decontamination) of items that come into contact with the bodily substances of people or animals such that they are substantially “substance free” (of, e.g., viruses, bacteria, detergent, sterilant, lipids, etc.) represent an immense and ongoing challenge. This challenge has been underscored by a recent article entitled “Widely used sterilizer under attack” (published in Jan. 21, 2003 edition of the newspaper USA Today). The article describes a fatal outbreak of bacterial infection that was linked to the improper sterilization of hospital bronchoscopes. Despite the hospital's use of one of the most popular sterilizing systems, tests performed by the Centers for Disease Control and Prevention found bacteria on the system's water filters and in its rinse water. This and other infection outbreaks has led to continuing controversy over how best to clean and sterilize used endoscopes.

The contaminants typically found on tubular or “lumened” medical items, such as endoscopes, are especially difficult to remove. In addition to fecal mater, loose cellular debris, blood and blood products, viruses, and bacteria, an endoscope can be coated with various hydrophobic films, such as “biofilm” material. A biofilm typically comprises cells, both dead and alive, cell debris and extracellular polymer substances. Once biofilm is formed by microorganisms (including bacteria, fungi, and protozoans), these microorganisms can colonize and replicate on the interior surfaces of tubing, forming a protective slime layer known as a “glycocalyx” that is especially difficult to remove.

Merely soaking endoscopes in a sterilant or detergent is unacceptable since numerous pockets exist within the tubing where the sterilant or detergent cannot reach effectively, which leaves areas of contamination within the endoscope. Moreover, with the prevalence of highly contagious diseases such as hepatitis B and C and Acquired Immune Deficiency Syndrome, reliable sterilization or disposal of all used medical tools seemingly becomes mandatory. Yet, while many medical instruments today are routinely cleaned, disinfected, and reused, experts in the field recently have warned that some of the more difficult to clean and sterilize medical items are putting people at risk.

After sterilization, endoscopes typically are rinsed with water filtered down to the 0.2 micron (200 nanometer) level. Unfortunately, many viruses, endotoxins, and prions are smaller than 200 nanometers, meaning that they can remain in the water even after filtration. Also, water and water filters are known sources of contamination. Even more troubling, however, is the statement by one expert that “there are no independent data in the medical literature that support the production of sterile water (a biological endpoint defined as containing fewer than 10⁻⁶ CFU/ml or fewer than 5 endotoxin units/ml) by passing unprocessed water (that is, un-sterilized water, such as water that flows though a hospital's tap) through a bacterial (e.g., 0.1 or 0.2 micron) filtration system” (See Comments by L. Muscarella (Custom Ultrasonics) on AAMI TIR7:1999, Chemical Sterilants and Sterilization Methods: A Guide to Selection and Use, downloaded from the website myendosite.com). Moreover, there is no currently available system that monitors the biological content of filtered water to insure its sterility when used in conjunction with medical item cleaning or sterilization apparatuses. Finally, having to add additional sterilization steps and/or use sterilized (e.g., autoclaved) water becomes tedious and expensive.

Many attempts to improve sterilization apparatus and methods have been attempted. For example, a variety of gas sterilization methods has been investigated in the past. Methods using ethylene oxide and other disinfecting gases are widely used for sterilizing a wide range of items, from contact lenses to surgical instruments.

A sterilizing method must effectively kill all organisms, including spores, without damage to the article or goods being sterilized. However, many disinfecting gases which meet this criterion, such as ethylene oxide, have been recognized to expose workers and the environment to safety hazards.

The use of gas plasma to sterilize containers was suggested in U.S. Pat. No. 3,383,163. Plasma is an ionized body of gas which may be generated by the application of power from different sources. The ionized gas will contact microorganisms on the surfaces of the items to be sterilized and effectively destroy the microorganisms.

Plasma gas sterilizer systems as described in U.S. Pat. Nos. 3,851,436 and 3,948,601 include separate plasma Radio Frequency (RF) generation chambers and sterilizing chambers. A gas plasma produced in the plasma generating chamber with argon, helium, nitrogen, oxygen or xenon is passed into a separate sterilization vacuum chamber containing the articles to be sterilized.

While sterilization using gas and gas plasma may be effective for some applications, it is often the case that the sterilization procedure must be carried out quickly due to degradation of the sterilant and/or damage to the item being sterilized. Moreover, problems involve chemical residue left on items and environmental toxicity can complicate the use of many gases.

So-called “push/pull reprocessing systems” are automatic apparatuses that include a chamber containing a baffle with one or more openings through which water (or another fluid) surges in a synchronous fashion back-and-forth (hence “push/pull”) through the opening or openings in the baffle. When soiled items, such as endoscopes and other lumened instruments, are placed within an opening in the baffle, fluid also surges upon and through them. Accordingly, a back-and-forth “scrubbing action” is created by the surging fluid the contacts any accessible surface on an item, including any lumen or lumens.

For example, U.S. Pat. No. 5,711,921 by Langford discloses a medical apparatus cleaning mechanism that includes a container having a first chamber and a second chamber, with the container adapted to accept a medical instrument such that a first portion of the medical instrument lies in the first chamber and a second portion of the medical instrument lies in the second chamber. Pumping means then simultaneously increase fluid pressure within the first chamber of the container while decreasing fluid pressure within the second chamber until the cycle is reversed, i.e., the fluid pressure in the second chamber increases while the fluid pressure in the first chamber synchronously is decreased. The end result is a linear flow that is generated by the simultaneous modulation of fluid pressure in each chamber in opposite directions (see FIG. 1).

While such systems are known to provide superb cleaning and disinfection with fluids, having a sterilant passively contact an item or moving a single quantity of sterilant over an item may not always provide satisfactory results, particularly when the sterilant has a short “half life” or otherwise loses effectiveness quickly.

Moreover, once an item has undergone reprocessing (including sterilization), it must either be used immediately or packaged under sterile conditions for storage and later use. Thus, the maintenance of sterile conditions can be problematic if items are packaged outside of the reprocessor. However, the use of a “back and forth” motion to sterilize an item that has been placed within packaging may actually generate shear forces that can damage that packaging.

Accordingly, it would be desirable to provide a sterilizing apparatus and method that carries out effective sterilization quickly and thoroughly, while reducing undesirable effects.

### SUMMARY OF THE INVENTION

The invention generally involves methods and apparatus for reprocessing items, including those with lumens. According to one aspect of the invention, an item is cleaned within a push/pull reprocessing apparatus, contained within sterilizable packaging, and sterilized within the reprocessing apparatus through the introduction of a sterilant, the flow of which includes fresh or new sterilant at one or more times during the sterilizing cycle. The apparatus of the invention includes one or multiple chambers, within which cleaning and/or sterilizing take place.

In one embodiment, the invention includes a plasma sterilizer that employs uncharged, highly reactive free radicals, atoms, and excited molecules of a gas mixture to sterilize articles. According to this aspect of the invention, a sterilizer apparatus includes a sterilizing chamber, a plasma generating chamber/mixing tank adapted to allow a gas mixture streaming therethrough, means for ionizing the gas mixture in the plasma generating chamber/mixing tank, and a plasma distribution means for distributing downstream plasma gas products to the sterilizing chamber.

A removable baffle plug of the invention is provided to allow for the placement of different sized items within the baffle, to modulate the pressure between parts of the chamber on either side of the baffle, and to provide information about what is being placed in the chamber through the use of radio frequency identification devices.

In accordance with the above methods, there is provided new and improved apparatus for sterilizing an item. In one embodiment, the method for sterilizing includes the steps of placing am item within an enclosed chamber, introducing a sterilant into the chamber, and sterilizing the item while fresh sterilant is introduced into the chamber at least once after the sterilant is initially introduced.

Various other purposes and advantages of the invention will become clear from its description in the specification that follows. Therefore, to the accomplishment of the objectives described above, this invention includes the features hereinafter fully described in the detailed description of the preferred embodiments, and particularly pointed out in the claims. However, such description discloses only some of the various ways in which the invention may be practiced.

### BRIEF DESCRIPTION OF THE DRAWINGS

FIG. 1 depicts in a cross-sectional view a “push-pull” reprocessing apparatus of the prior art.

FIG. 2 schematically illustrates a first apparatus and method embodiment of the invention.

FIG. 3 schematically illustrates a second apparatus and method embodiment of the invention.

FIG. 4 schematically illustrates a third apparatus and method embodiment of the invention.

FIG. 5 schematically illustrates an enlarged view of section X taken from FIG. 4.

FIG. 6 schematically illustrates the same enlarged view as FIG. 5 but with the packaging around the item opened.

FIG. 7 shows a removable baffle plug of the invention.

### DETAILED DESCRIPTION OF THE PREFERRED EMBODIMENTS

The invention relates generally to methods and apparatus for sterilizing items that involve placing one or more items within an enclosed chamber, introducing a sterilant into that chamber, and sterilizing the item while fresh sterilant is introduced into chamber at least during the sterilizing process. By flowing fresh sterilant upon an item at more than one point during the sterilizing process, more effective interaction with the surfaces of the item can be attained. Moreover, according to the invention, a sterilant is flowed in a manner (e.g., pulsed, unidirectional, bi-directional, etc.) that covers surfaces of an item better than passive soaking or diffusion, while reducing the chances of damage to an item or item packaging caused by shear forces.

One embodiment of the invention includes an apparatus for sterilizing an item via a push/pull system. The apparatus may be provided with or without a dispenser of sterilizable packaging coupled to the reprocessing chamber such that the sterility of items may either be “transient” or portable.

An apparatus of the invention may contain a single chamber, within which both cleaning and sterilization takes place, or a cleaning chamber and a separate sterilization (before or after an item is packaged) chamber.

The terms “push/pull apparatus,” “push/pull system,” “cleaner/sterilizer,” “push/pull reprocessor,” “reprocessor,” and “reprocessing” as used throughout the specification are meant to be synonymous with the use of a push-pull apparatus (such as the Langford IC Systems, Inc. Manzi Mach 1) that cleans items (e.g., endoscopes, dental appliances, surgical instruments and the like) by surging fluid back-and-forth upon the accessible surfaces of these items.

The term “bi-directional” means flowing in two directions at once within the chamber of the invention.

As used herein, the term “plasma” is intended to include any portion of the gas or vapors which contains electrons, ions, free radicals, and the like produced as a result of an applied electrical field, including any accompanying radiation that might be produced. While radiation in the radio frequency range is most commonly applied, a broad frequency range may be used.

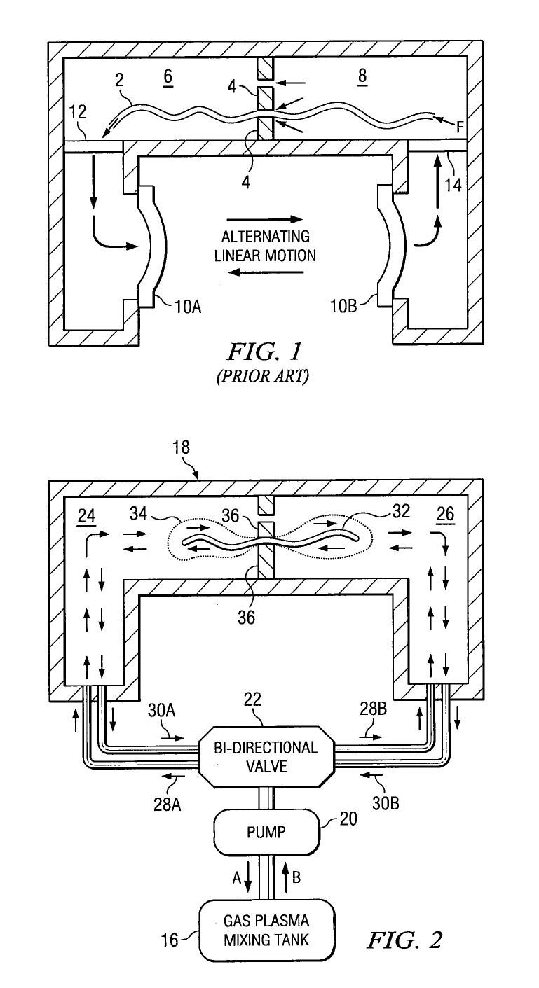

Turning to the figures, wherein like numbers designate like features, FIG. 1 depicts a “push-pull” reprocessor of the prior art. An item with a lumen 2 is secured within an opening of a baffle 4 that divides the reprocessor into a first chamber 6 and a second chamber 8. When diaphragms 10A and 10B are simultaneously activated in opposite directions as shown, fluid F flow through and around the lumen 2. By reversing the direction of the diaphragms 10A and 10B in unison, a linear or unidirectional flow is created first one way and then the other, resulting in a periodic shear force or “scrubbing action.”

FIG. 2 illustrates in simple schematic form a first embodiment of the invention. Unlike the linear flow of an initial quantity of “old” sterilant and “push/pull” action created by the apparatus of FIG. 1, this embodiment of the invention bi-directionally flows fresh sterilant into chamber 18. Thus, for example, mixing tank 16 housing gas plasma is distributed into chamber 18 via a pump 20 and a bi-directional valve 22. The bi-directional valve 22 conveys plasma into first chamber 24 and second chamber 26 at the same time, resulting in a bi-directional flow (indicated by arrows 28A and 28B for inflow and arrows 30A and 30B for outflow when the pump is reversed) over item 32 and gas permeable packaging 34. Reversing the pump at desired intervals (e.g., 2 seconds as shown) allows for at least partial evacuation of “old” sterilant while introducing fresh or “new” sterilant upon the item 32.

While a chamber 18 containing a baffle 36 is shown in this and other embodiments, it should be understood that the invention may include a chamber with no baffle and into which an item is simply placed or secured to a clamp or other structure within the chamber. Moreover, the invention is not limited to gas plasma. Other sterilants may include, for example, ethylene oxide or steam.

In view of the above apparatus and explanation, a method for sterilizing an item encompasses: placing an item 32 within an opening in baffle 36 such that the item 32 extends into the first chamber 24 and second chamber 26, introducing the sterilant into the first and second chambers such that the sterilant is made to flow bi-directionally (28A, 28B, 30A, 30B) in the chambers, and sterilizing the item while introducing fresh sterilant (symbolized by arrows A and B) into the chamber 18 at least once during a sterilizing cycle.

The methods of the invention are especially suitable for sterilization of an item inside a gas-permeable sleeve or packaging 34, because the lack of linear flow or substantial “back and forth” motion reduce the chances that the packaging will be damaged or lost (through, for example, being impaled by the item 32 or by sliding off as a result of being pulled in one direction). In the embodiment shown in FIG. 2, this is accomplished by introducing and evacuating the sterilant on each side of said baffle to provide a continuous bi-directional flow upon item 32.

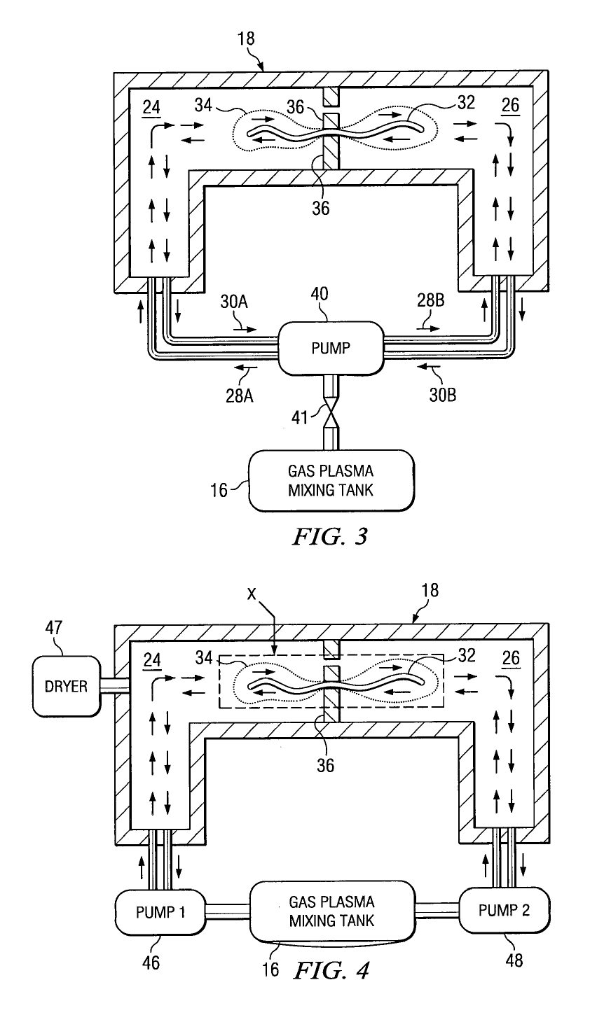

In another embodiment of the invention pictured in FIG. 3, gas plasma from tank 16 is alternately introduced on each side of baffle 36 through reversing the one-way pump 40 (during periods in which valve 41 is open). Because sterilant is introduced into only one of chambers 24 or 26 at a time, this embodiment initially appears to provide a “uni-directional” flow of fresh sterilant suitable for sterilizing an item with a lumen. However, as the one-way pump is reversed periodically, the net effect is have gas flowing within the apparatus 18 dynamically to both chambers, with new sterilant being introduced as desired by opening valve 41.

FIG. 4 illustrates a third embodiment of the invention. Here, the bi-directional flow of sterilant from mixing tank 16 is created by a plurality of pumps 46 and 48. Similar to the one-way pump show in FIG. 3, a net bi-directional flow effect is created by alternating between pump 46 and 48. Moreover, pumps 46 and 48 can be used at the same time to create a “pulsing” effect, whereby waves of fresh sterilant are introduced into chamber 18. The pumps may provide a continuous flow of fresh sterilant or may pump the existing sterilant (though the use of valves or a bypass (not shown) with the mixing tank 16), with new sterilant being introduced as desired.

In some applications, it may be preferred to dry an item prior to introducing a sterilant. Thus, drying means 47 is connected to chamber 18 to provide heated air. Alternatively, alcohol (e.g., 70% ethanol) or a vacuum can be provided in order to dry item 32.

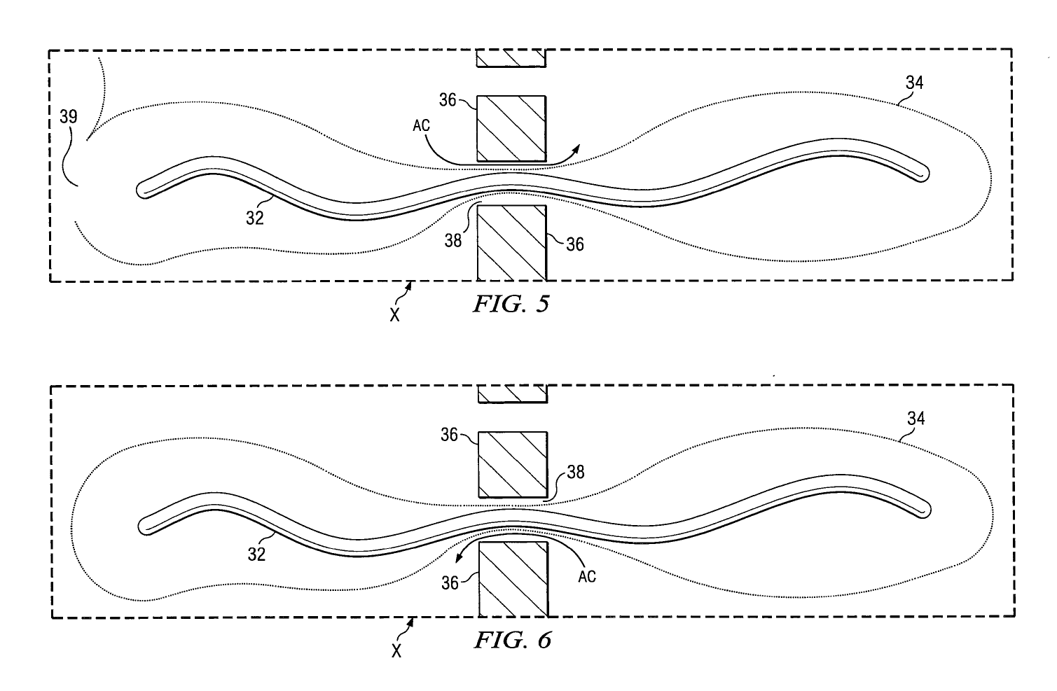

FIGS. 5 and 6 are greatly enlarged views of section X of FIG. 4. Thus, opening 38 in baffle 36 is more clearly seen. Preferably, the item 32 is contained in a bag 34 having a sealable end 39 (e.g., such as by adhesive) and is placed into opening 38. Also preferably, the item 32 and opening 38 have a “wet fit,” meaning that the friction between the opening and the item is such that fluid (including a gas) can flow between different sides of the baffle 36 as shown by arrow AC.

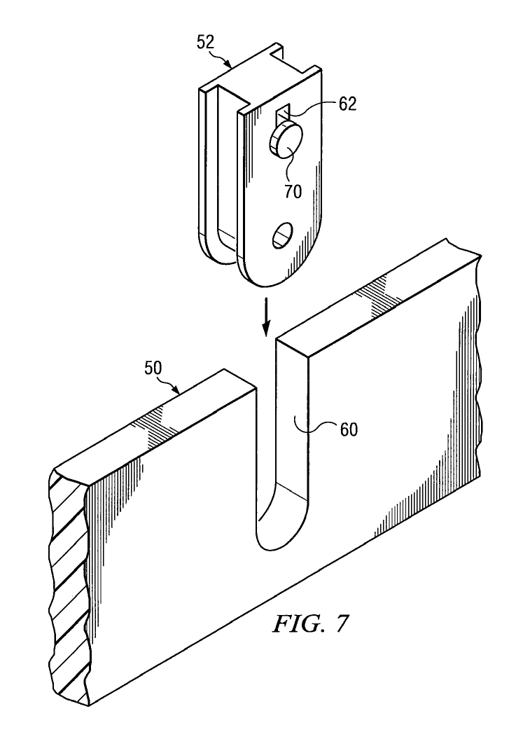

To control the flow between different sides of baffle, and to make the baffle adaptable to different sized items, one or more removable plugs may be used with the invention. Turning to FIG. 7, baffle 50 has a removable plug 52 that is disposed within notch 60 and in sealed arrangement therewith. Thus, plugs with different sized openings 62 may be utilized for different sized items. Moreover, one or more plugs 52 may be removed or filled in to change the flow dynamics between chambers on either side of the baffle.

Also, radio frequency (RF) transmitters and receivers can be used in conjunction with the plug of the invention. Thus, RF device 70 disposed upon plug 52 can be used to identify a compatible item also containing a RF device. For example, RF device 70 could identify a bronchoscope placed within the opening 62 of plug 52 and transmit such information to the operator of the sterilizing apparatus so that a particular program or cycle could be employed.

Within the following examples, endoscopes or other medical or dental instruments will be used as an example of an item or instrument to be reprocessed. However, the inventor contemplates use of the invention with any tubular item, as well as a variety of other items such as circuit boards, cosmetic instruments, food preparation instruments, and other items in which reliable cleaning and sterility are desirable or required.

### EXAMPLE 1

The purpose of this test is to document the results of engineering characterization testing performed on a automatic endoscope reprocessor, the Langford I.C. Systems Sterilizer Cleaner (see U.S. Pat. No. 5,906,802 for layout and guidance in the use of this reprocessor). Testing was performed on a Cleaner, Sterilizer Breadboard.

The biopsy lumen of three bronchoscopes were loaded with Birmingham Soil (much more than required by FDA test standards) and inoculated with pathogens from an American Society of Test Methods kit. The scopes were left sitting for a 24 hour time period to permit some drying. Using the same Langford I.C. Systems Sterilizer Cleaner liquid-displacement settings as described, each colonoscope was subjected to one wash cycle at 10 psi for 5 min with a use concentration of 2.5% of enzymatic cleaner in 10 liters of water. The preferred rate of “liquid displacement” (i.e., the back-and-forth liquid cycling rate in the item-washing chamber of the Sterilizer Cleaner) is 1 gallon per 2 seconds.

Upon completion of the cleaning cycle, the bronchoscope is dried for 5 minutes with heated air and the sterilization cycle takes place by flowing gas plasma for 5-30 minutes upon the bronchoscope. At least once after the beginning of the sterilizing cycle, fresh gas plasma is pulsed into the chamber to better ensure all surfaces are contacted with active sterilant.

### EXAMPLE 2

A surgical item is placed in a multi-chamber reprocessor and cleaned as above. The item is then packaged and sealed in a cellulose envelope and transferred to a chamber separate from the cleaning chamber. The separate chamber includes a fluid port to bi-directionally inject gas into the chamber, with a pump alternately applying positive pressure to the chamber and a vacuum to evacuate the chamber. The separate chamber includes radio frequency electrodes to generate the requisite radio frequency signal. The plasma is generated by evacuating the chamber, introducing a gas or vaporized liquid and turning on the power to the electrodes. The plasma is generated in the present process in the same manner as in known prior art plasma sterilization system (e.g., U.S. Pat. No. 4,643,876). The surgical item is exposed from 5-30 minutes to the plasma.

By way of example, hydrogen peroxide is injected in the form of an aqueous solution of hydrogen peroxide containing from about 3% to 20% by weight of hydrogen peroxide. The concentration of hydrogen peroxide vapor in the chamber may range from 0.05 to 10 mg of hydrogen peroxide per liter of chamber volume. A concentration of 0.125 mg per liter is the minimum preferred concentration of hydrogen peroxide. Air or an inert gas such as argon, helium, nitrogen, neon or xenon may be added to the chamber with the hydrogen peroxide to maintain the pressure in the chamber at the desired level. The hydrogen peroxide solution may be injected in one or more separate injections. Since the hydrogen peroxide is decomposed into non-toxic products during the plasma treatment, no additional steps are required to remove residual hydrogen peroxide from the sterilized object or its packaging prior to use of the object.

Alternatively, if sterilization of a lumened instrument (e.g., a bronchoscope) is desired, the above method could be modified for use in a push/pull reprocessor to flow gas plasma upon all accessible surfaces, including the exterior and through the lumen of the scope (before or after any packaging of the item takes place). “Unidirectional” flow (with occasional reversal of direction) would be employed to urge the gas plasma (or other vapor-phase sterilants, such as steam) to effectively permeate through the entire lumen.

Various changes in the details and components that have been described may be made by those skilled in the art within the principles and scope of the invention herein described in the specification and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products. All references cited in this application are hereby incorporated by reference herein.

What is claimed is:

1. A method for sterilizing an item, comprising:
(a) placing said item within an enclosed chamber;
(b) introducing a sterilant into said chamber; and
(c) sterilizing the item, wherein fresh sterilant is introduced into said chamber at least once after step (b).

2. The method of claim 1, wherein step (c) comprises providing a continuous flow of fresh sterilant.

3. The method of claim 1, wherein step (c) comprises providing a pulsed flow of fresh sterilant.

4. The method of claim 1, wherein step (c) further comprises at least partially evacuating previously introduced sterilant from the chamber before introducing fresh sterilant.

5. The method of claim 1, wherein step (c) further comprises at least partially evacuating previously introduced sterilant from the chamber simultaneously while introducing fresh sterilant.

6. The method of claim 1, wherein step (a) further includes providing a baffle in said chamber and securing said item within said baffle.

7. The method of claim 1, wherein step (c) further comprises providing a bi-directional flow of fresh sterilant within said chamber.

8. The method of claim 6, wherein step (c) further comprises providing a bi-directional flow of fresh sterilant within said chamber.

9. The method of claim 1, further including providing gas-permeable packaging around said item.

10. The method of claim 9, wherein providing said packaging comprises providing cellulose with a sealable end.

11. The method of claim 1, wherein said sterilant is selected from the group consisting of steam, ethylene oxide, and gas plasma.

12. The method of claim 1, further including the step of drying said item prior to introducing said sterilant.

13. The method of claim 12, wherein said drying step includes providing one or more selected from the group consisting of forced air, alcohol, and a vacuum.

14. An apparatus for sterilizing an item, comprising:
an enclosed chamber;
a container housing a sterilant in flowable connection with said enclosed chamber; and
pumping means for introducing said sterilant into said chamber, wherein said pumping means is adapted to introduce additional fresh sterilant at least once during a sterilization cycle.

15. The apparatus of claim 14, wherein said pumping means comprises a pump adapted to circulate a continuous flow of fresh sterilant into said enclosed chamber.

16. The apparatus of claim 14, wherein said pumping means comprises a pump adapted to circulate a pulsed flow of fresh sterilant into said enclosed chamber.

17. The apparatus of claim 14, wherein said pumping means comprises a pump adapted to at least partially evacuate previously introduced sterilant from the chamber before introducing fresh sterilant.

18. The apparatus of claim 14, wherein a bi-directional valve is disposed between said pumping means and said enclosed chamber.

19. The apparatus of claim 14, wherein said pumping means includes a reversible pump.

20. The apparatus of claim 14, wherein said pumping means includes at least two pumps.

21. The apparatus of claim 14, further including a baffle in said enclosed chamber.

22. The apparatus of claim 21, wherein said pumping means is adapted to bi-directionally flow fresh sterilant within said enclosed chamber on both sides of said baffle.

23. The apparatus of claim 14, wherein said enclosed chamber further includes an item contained within gas-permeable packaging.

24. The apparatus of claim 23, wherein said packaging comprises cellulose with a sealable end.

25. The apparatus of claim 14, wherein said sterilant is selected from the group consisting of steam, ethylene oxide, and gas plasma.

26. The apparatus of claim 14, further including a means for drying in flowable connection with said enclosed chamber.

27. The apparatus of claim 26, wherein said means for drying includes one or more selected from the group consisting of forced air, alcohol, and a vacuum.

28. The apparatus of claim 14, wherein said apparatus comprises a push/pull reprocessing apparatus having a baffle.

29. The apparatus of claim 28, wherein said push/pull reprocessing apparatus contains a single chamber within which both cleaning and sterilization takes place.

30. The apparatus of claim 21, wherein said baffle further includes a removable plug.

31. The apparatus of claim 28, wherein said baffle further includes a removable plug.

32. The apparatus of claim 30, wherein said plug further includes a radio frequency device.

33. The apparatus of claim 31, wherein said plug further includes a radio frequency device.
